(19) United States
(12) Patent Application Publication
(10) Pub. No.: US 2026/0363785 A1
(43) Pub. Date: Nov. 19, 2026

(54) APPLICATION OF COMPUTER VISION TO ENABLE REAL-TIME DIGITAL MEASUREMENTS AND TARGETING DURING ARTHROSCOPY

(71) Applicants: The General Hospital Coporation, Boston, MA (US); Pictus AI LLC, Del Mar, CA (US)

(72) Inventors: Miho Tanaka, Boston, MA (US); Kartik Mangudi Varadarajan, Boston, MA (US)

(21) Appl. No.: 19/126,613

(22) PCT Filed: Nov. 7, 2023
(86) PCT No.: PCT/US2023/078944

§ 371 Date: May 1, 2025

Related U.S. Application Data
(60) Provisional application No. 63/382,714, filed on Nov. 7, 2022.

(51) Int. Cl. A61B 34/20; A61B 34/10; A61B 90/00; G06T 7/60; G06T 7/70

(52) U.S. Cl. CPC A61B 34/20; A61B 34/10; A61B 90/06; A61B 90/361; G06T 7/60; G06T 7/70; A61B 2034/107; A61B 2034/2065; A61B 2090/061; G06T 2207/30004; G06T 2207/30204

(57) ABSTRACT

A system for performing digital measurements during a medical procedure comprises: a camera to capture image data corresponding to a treatment site; a display; an instrument placed at the treatment site, the instrument including a first section having a first dimension between a first boundary and a second boundary; and a processor configured to execute instructions to: (i) receive real-time image data from the camera, the real-time image data including a portion of the instrument, (ii) detect, in the real-time image data, a profile of the instrument, (iii) identify, on the instrument, the first boundary and the second boundary, (iv) determine a real-time measurement between a first set of points of the real-time image data, the real-time measurement being determined based in part on the first dimension, (v) generate a visualization based on the real-time measurement, and (vi) display, at the display, the visualization and the real-time image data.

### CROSS-REFERENCE TO RELATED APPLICATIONS

This application is based on, claims benefit of, and claims priority to U.S. Application No. 63/382,714 filed on Nov. 7, 2022, which is hereby incorporated by reference herein in its entirety for all purposes.

### STATEMENT REGARDING FEDERALLY SPONSORED RESEARCH

Not applicable.

### BACKGROUND

### 1. Technical Field

This invention relates, in general, to systems and methods for enabling real-time, digital on screen measurements and targeting during a medical procedure such as arthroscopic surgery.

### 2. Description of the Related Art

Approximately 1,770,000 arthroscopic procedures are performed each year in the United States alone. [Ref. 1] The initial part of any arthroscopic procedure includes diagnostic assessment of the joint to confirm and describe the existing injuries or lesions, as many prognostic and therapeutic outcomes stem from proper classification of lesions based on their size and location. The procedure then typically involves anatomically repairing or reconstructing the injured or diseased tissue, for which fixation devices (implant) or reconstructive tissue grafts are positioned based on visualization of known anatomic landmarks and intraoperative surgical measurements.

Technical error in the position of the graft or implant has been reported in arthroscopic knee procedures to account for up to 25-70% of complications and the need for subsequent revision surgery, with variations in position as little as few millimeters accounting for such errors. [Ref. 2, 3] As an example, after anterior cruciate ligament (ACL) surgery, which is performed over 250,000 times yearly in the US, revision surgery occurs at a rate of 10-15% and the contribution of surgical/technical error is reported to be around 70% of such cases. [Ref. 4, 5] With the current healthcare burden of ACL injuries and treatments alone estimated to be over $7.6 billion per year, reduction of technical error could lead to a significant improvement in healthcare costs, as well as in patient outcomes and the additional long term health effects associated with decreased knee function and mobility. [Ref. 6] Similar associations between technical variations and surgical outcomes have been identified in the cumulative millions of additional arthroscopic procedures that are performed in the knee as well as the shoulder, elbow, wrist, hip and ankle. Despite the increasing complexity of procedures that can be completed arthroscopically, and the need for surgical precision to attain good outcomes, the ability to perform measurements and targeting (i.e., planning the position of fixation devices or reconstructive tissue grafts) arthroscopically in the joint remains limited. Accurately obtaining measurements to describe the size of a lesion or to determine the optimal position for implant placement can normally be performed with a ruler during traditional open surgery. However, this is not possible during arthroscopy due to the limitations in orienting measurement devices through standard surgical portals that are used to access the joint.

Currently, measurements performed to describe a lesion or to identify optimal implant position are approximated by placing an instrument of known dimensions, (such as a hook probe) in the view of the arthroscope camera, and the user/surgeon estimating the distances relative to this instrument (see FIG. 1). For example, when the tool is a probe hook, the known height of hook (hook height), known fixed spacing between bands on the probe body (band gap), and known cylindrical probe diameter, can be used for estimating distances (see FIG. 2). However, the challenges with this current approach can be appreciated by reviewing a few examples from FIG. 3. In FIG. 3 Panel B, the probe cannot be oriented parallel to the width of the cartilage lesion, and the probe bands are not clearly visible; therefore, accurate measurement of the cartilage lesion in this case would be challenging. In FIG. 3 Panels D-F, the surgeon needs to measure gaps between two bones. In this case, the hook could be utilized as a measurement reference. However, in FIG. 3 Panel D and FIG. 3 Panel E, the hook height is smaller than the gap height, which implies that the surgeon would have to mentally estimate how much taller or shorter the gap height is relative to the hook height. In FIG. 3 Panel F, the probe hook cannot be positioned vertically so the surgeon would have to perform trigonometric calculations to estimate the gap height.

Such challenges can lead to lack of precision and variability in measurements, which can be particularly problematic when the subsequent step in surgery depends on selection of a graft or implant of an appropriate size. Additionally, lack of precise measurements can lead to inaccurate diagnosis/classification, or errors in positioning of implants or grafts, which can subsequently have negative consequences on patient outcomes. Furthermore, the need to perform mental estimates/calculations creates unnecessary burden for the surgeon, and makes it less likely that accurate data is captured in the operative notes, which impacts the ability to improve clinical guidelines and treatment protocols.

While arthroscopic camera systems currently do not allow for real-time on-screen digital measurements, clinical research studies utilizing post-hoc analysis of arthroscopic images have shown digital measurements to have value in the diagnosis and classification of ligament injuries, which can accurately determine the need for, and assess the efficacy of, surgical procedures. For example, in [Ref. 7], Tanaka et al. scaled/calibrated arthroscopic images post-surgery based on known dimensions of hook probe, and then manually made digital point-to-point distance measurements at locations “L” and “M” in FIG. 4 Panel A. Similarly, in [Ref. 8], Han et al. manually measured probe hook height and angles on images post-surgery, and then applied trigonometric formulas to calculate distances (see FIG. 4 Panel B).

Accordingly, there is a need for systems and methods that enable digital measurements on a display screen of the system in real-time during a medical procedure such as arthroscopic surgery.

### SUMMARY OF THE DISCLOSURE

The present disclosure meets an important clinical need in medical procedures by providing systems and methods for enabling real-time, digital on screen measurements and targeting during a medical procedure such as arthroscopic surgery.

The present disclosure describes systems and methods, for enabling real-time digital measurements and targeting during a medical procedure such as arthroscopic surgery. At a high level, image processing methods either alone or in combination with data from positional sensors are used to detect the shape, key-points, and/or features of an object such as an instrument visible in the arthroscopy video. The detected shape, key-points, and features are then used to track the instrument and present real-time on-screen information (such as an extendable virtual ruler, or variable virtual graft/drill diameter), which can be used for making quick and accurate digital measurements, and targeting decisions during arthroscopy surgery. The image processing methods, include both classical and modern deep learning-based computer vision methods. The projected locations of instrument key-points on the image can be predicted via computer vision models, and combined with the predicted segmentation (via PnP matching, and 3D-to-2D matching) to predict the six degree-of-freedom (6DOF) pose (location/orientation) of the instrument. Once the 6DOF pose is known, it is combined with stored 3D geometric data of the instrument to place and appropriately scale the virtual overlays on the correct location on the image via coordinate transformations.

In one aspect, the disclosure provides a system for performing digital measurements during a medical procedure in a subject. The system can include a camera, a display, an instrument, at least one memory, and at least one processor. The camera can be positionable to capture image data corresponding to a treatment site in a subject. The instrument can be dimensioned to be placed at the treatment site, and can include at least a first section having a first dimension between a first boundary and a second boundary. The at least one memory can have stored thereon a configuration corresponding to the instrument, the configuration including the first dimension. The at least one processor can be in electrical communication with the camera and the at least one memory. The at least one processor can be configured to execute instructions stored in the at least one memory to: receive real-time image data from the camera, the real-time image data including at least a portion of the instrument; detect, in the real-time image data, a profile of the instrument; identify, on the instrument, the first boundary and the second boundary; determine a real-time measurement between a first set of points of the real-time image data, the real-time measurement being determined based at least in part on the first dimension; generate a visualization based on the real-time measurement; and display, at the display, the visualization and the real-time image data.

In one embodiment of the system, the first dimension is a diameter of the instrument. In one embodiment of the system, the first boundary and the second boundary are visually-identifiable markings along the instrument.

In one embodiment of the system, the instrument includes a removable sleeve, wherein the first boundary and the second boundary are defined on the removable sleeve.

In one embodiment of the system, the visualization includes a first digital ruler, wherein the first digital ruler is overlaid onto the real-time image data at the display.

In one embodiment of the system, the instrument comprises an awl.

In one embodiment of the system, the at least one processor is further configured to execute instructions stored in the at least one memory to identify, in the real-time image data, a distal tip of the instrument. In one embodiment of the system, a placement of the visualization on the display is based at least in part on a position of the distal tip. In one embodiment of the system, the real-time measurement is a measurement of a radius extending from the distal tip. In one embodiment of the system, the first set of points comprises a first point corresponding to a position of the distal tip at a first time, and a second point corresponding to a position of the distal tip at a second time, wherein the first measurement is a distance between the first point and the second point.

In one embodiment of the system, the instrument includes a first linear portion and a second linear portion, the second linear portion being substantially perpendicular to the first linear portion, the second portion defining a second dimension between a first end and a second end, wherein the configuration includes the second dimension, and wherein the at least one processor is further configured to execute instructions stored in the at least one memory to determine a second real-time measurement between a second first set of points of the real-time image data, the second real-time measurement being determined based at least in part on the second dimension.

In one embodiment of the system, the at least one processor is further configured to execute instructions stored in the at least one memory to receive an instrument input, and, based on the instrument input, select, from a plurality of instrument configurations stored in the at least one memory, the configuration.

In one embodiment of the system, the medical procedure is an arthroscopic knee procedure.

In one embodiment of the system, the instrument includes a first three-dimensional key point, wherein determining the real-time measurement includes predicting a location of the first three-dimensional key point on the real-time image.

In one embodiment of the system, determining the real-time measurement includes minimizing a difference between the profile of the instrument and a two-dimensional projection of a three-dimensional model of the instrument, and, based on the two-dimensional projection, determining a pose of the instrument relative to the camera, wherein the real-time measurement is based on the pose.

In one embodiment of the system, the pose of the instrument is a 6 degree of freedom pose.

In yet another aspect, the disclosure provides a method for performing digital measurements during a medical procedure in a subject. The method comprises: receiving a first image, the first image including a visual representation of a first instrument and a tissue of a subject; detecting, within the first image, a profile of the first instrument, including an outer edge of the first instrument; comparing the profile of the first instrument to a two-dimensional projection of a three-dimensional model of the first instrument to determine a pose of the instrument relative to the camera; identifying a first key feature of the instrument; determining, based on the first key feature and the pose, a first dimension of the first image; generating an overlay including a first visualization based at least in part on the first dimension; and outputting, at a display, the overlay and the first image.

In one embodiment of the method, detecting a profile of the first image includes inputting the first image into a trained machine learning model, the trained machine learning model being trained on a first plurality of images, each of the first plurality of images including a first label identifying an instrument within the image, and a second label identifying a tissue within the image.

In one embodiment of the method, the first image is received in real-time from an arthroscope.

In one embodiment, the method further comprises providing, prior to receiving the first image, a plurality of two-dimensional projections of the instrument, and selecting a first two-dimensional projection of the plurality of two-dimensional projections based on a similarity between the profile of the first instrument and the first two-dimensional projection, wherein performing the smoothing operation includes altering the profile to increase a similarity between the profile and the first two-dimensional projection.

In one embodiment of the method, the first key point is a first band positioned along the instrument, wherein the first dimension is a known distance between the first band and a second band positioned along the first instrument.

In one embodiment of the method, the first visualization is a digital ruler, wherein a scale of the digital ruler is determined at least in part based on the first dimension.

In one embodiment, the method further comprises: receiving a second image, the second image including a visual representation of the first instrument and a tissue of the subject; identifying, in the second image, the first key point; determining, based at least partially on the first dimension, a measurement between a position of the first key point in the first image, and the first key point in the second image; and outputting, at the display, the measurement.

In one embodiment of the method, the medical procedure is an arthroscopic knee procedure. In one embodiment of the method, the tissue is tissue of a joint. In one embodiment of the method, the joint is a knee.

In still another aspect, the disclosure provides a computer system comprising instructions stored on a non-transitory computer readable medium. The instructions can cause at least one processor on a computer to: receive a first image, the first image including a visual representation of a first instrument and a tissue of a subject; detect, within the first image, a profile of the first instrument, including an outer edge of the first instrument; determine a predicted pose of the first instrument relative to the camera based on the profile; select a first two-dimensional projection of a three-dimensional model of the instrument based on the predicted pose; determine an actual pose of the instrument based at least in part on a comparison between the profile and the first two-dimensional projection; determine, based on the actual pose, a first dimension of the first image; generate an overlay including a first visualization based at least in part on the first dimension; and output, at a display, the overlay and the first visualization.

In one embodiment of the computer system, the tissue is tissue of a joint. In one embodiment of the computer system, the joint is a knee.

In one embodiment of the computer system, the instructions stored on the non-transitory computer readable medium further cause at least one processor on a computer to: select, based on the comparison between the profile and the first two-dimensional projection, a second two-dimensional projection, minimizing a difference between the profile and the second two-dimensional projection, wherein the actual pose corresponds to a model pose of the second two-dimensional projection.

In yet another aspect, the disclosure provides a method for performing digital measurements during a medical procedure in a subject. The method can include receiving a first image, the first image including a visual representation of a first instrument and a tissue of a subject. A profile of the first instrument, including an outer edge of the first instrument, can be detected in the first image. A smoothening operation can be performed on at least a first portion of the outer edge, wherein the smoothening operation at least partially alters a profile of the first instrument. A first key feature of the instrument can be identified. A first dimension of the first image can be determined, based on the first key feature. An overlay can be generated including a first visualization based at least in part on the first dimension. The overlay and the first image can be output to the display.

In one embodiment of the method, detecting a profile of the first image includes inputting the first image into a trained machine learning model, the trained machine learning model being trained on a first plurality of images, each of the first plurality of images including a first label identifying an instrument within the image, and a second label identifying a tissue within the image.

In one embodiment of the method, the first image is received in real-time from an arthroscope.

In one embodiment, the method further comprises: providing, prior to receiving the first image, a plurality of two-dimensional projections of the instrument, and selecting a first two-dimensional projection of the plurality of two-dimensional projections based on a similarity between the profile of the first instrument and the first two-dimensional projection, wherein performing the smoothing operation includes altering the profile to increase a similarity between the profile and the first two-dimensional projection.

In one embodiment of the method, the first key point is a first band positioned along the instrument, wherein the first dimension is a known distance between the first band and a second band positioned along the first instrument.

In one embodiment of the method, the first visualization is a digital ruler, wherein a scale of the digital ruler is determined at least in part based on the first dimension.

In one embodiment, the method further comprises: receiving a second image, the second image including a visual representation of the first instrument and a tissue of the subject; identifying, in the second image, the first key point; determining, based at least partially on the first dimension, a measurement between a position of the first key point in the first image, and the first key point in the second image; and outputting, at the display, the measurement.

In one embodiment of the method, the medical procedure is an arthroscopic knee procedure. In one embodiment of the method, the tissue is tissue of a joint. In one embodiment of the method, the joint is a knee.

In still another aspect, the disclosure provides a computer system comprising instructions stored on a non-transitory computer readable medium. The instructions can cause at least one processor on a computer to: receive a first image, the first image including a visual representation of a first instrument and a tissue of a subject; detect, within the first image, a profile of the first instrument, including an outer edge of the first instrument; perform a smoothening operation on at least a first portion of the outer edge, wherein the smoothening operation at least partially alters a profile of the first instrument; identify a first key feature of the instrument; determine, based on the first key feature, a first dimension of the first image; generate an overlay including a first visualization based at least in part on the first dimension; and output, at a display, the overlay and the first visualization.

In one embodiment of the computer system, the tissue is tissue of a joint. In one embodiment of the computer system, the joint is a knee.

These and other features, aspects and advantages of various embodiments of the present disclosure will become better understood with regard to the following description, appended claims, and accompanying Figures.

### BRIEF DESCRIPTION OF THE DRAWINGS

FIG. 1 shows measurements performed to describe a lesion or to identify optimal implant position by placing an instrument of known dimensions, (such as a hook probe) in the view of the arthroscope camera.

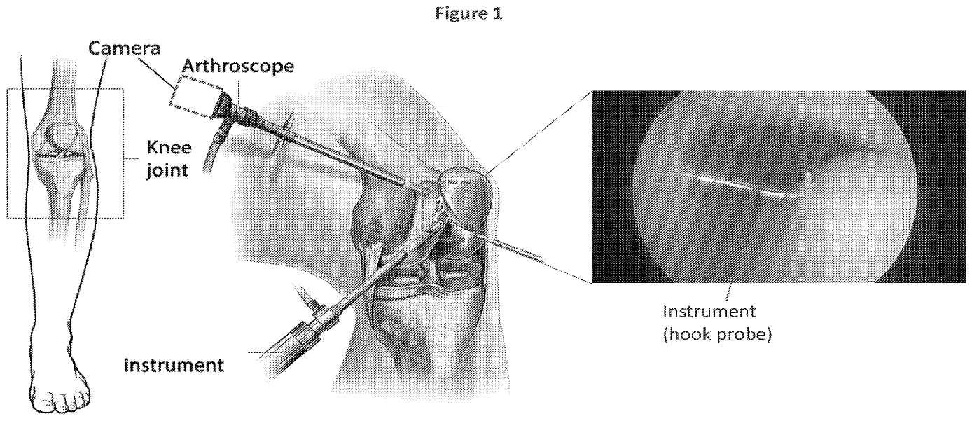

FIG. 2 is a detailed side view showing a hook probe used in estimating distances in a treatment site using an arthroscope camera.

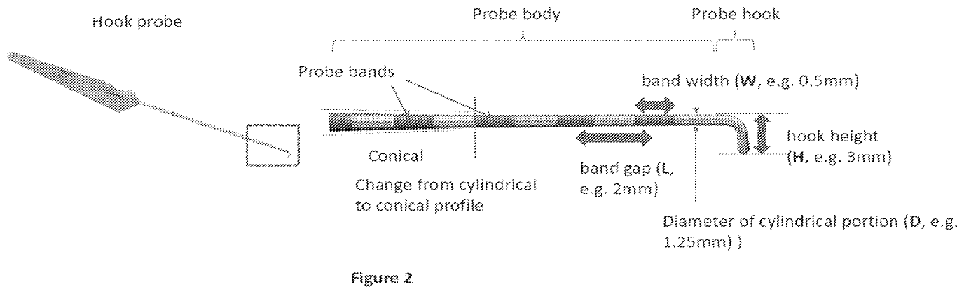

FIG. 3 shows measurements using arthroscopic images wherein: Panel A shows measurement of ligament tear width; Panel B shows measurement of cartilage lesion width; Panel C shows measurement of patella overhang; Panel D shows measurement of patella to trochlea gap height; Panel E shows measurement of femur to tibia gap height; and Panel F shows measurement of femur to tibia gap height.

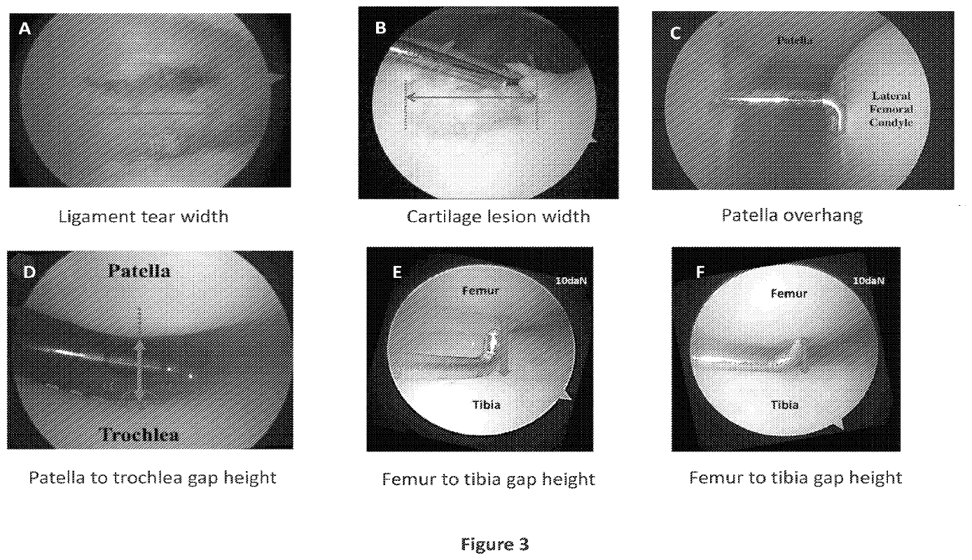

FIG. 4 shows: in Panel A, post-surgery knee measurements using the methods of Ref. 7; and in Panel B, post-surgery knee measurements using the methods of Ref. 8.

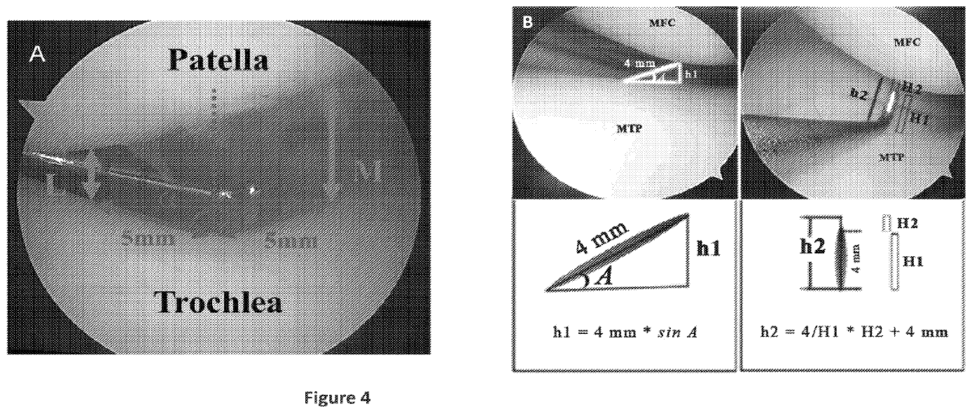

FIG. 5 shows a schematic of a non-limiting example system according to the present disclosure for performing digital measurements and/or targeting during a medical procedure in a subject.

FIG. 6 shows an example of real-time data about relative angular orientation of the instrument with respect to the camera/camera plane in three-dimensional space obtained via positional sensors that can be used in the system of FIG. 5

FIG. 7 shows: in Panel A, an example of an extendable virtual ruler overlay of a method of the present disclosure; in Panel B, examples of virtual area fitting circles/ellipses of a method of the present disclosure; and in Panel C, examples of virtual variable drill sizes of a method of the present disclosure.

FIG. 8 shows: in Panel A, a calibration image of a method of the present disclosure; in Panel B, predicted segmentation of a method of the present disclosure; and in Panel C, a predicted key-point heatmap of a method of the present disclosure.

FIG. 9 shows a step of a method of the present disclosure in which the models are trained to segment both the outer profile of the instrument and internal features such as the edges of bands.

FIG. 10 shows a step of a method of the present disclosure in which computer vision models are trained to detect the edges of internal features of the instrument. FIG. 10 Panel (A) shows a cropped probe image using segmentation mask, and FIG. 10 Panel (B) shows canny edge detection to identify edges of bands.

FIG. 11 shows steps of a method of the present disclosure in which computer vision models are trained to output predicted segmentation masks of the probe hook on the video frames wherein the predicted segmentation masks were then used to crop the image, and Sobel filter applied to detect the probe band edges. In FIG. 11, Panel A shows the original video frame; Panel B shows the detection of the probe hook, and predicted segmentation mask; Panel C shows the image cropped using predicted segmentation; and Panels D and E show the detections/calculations.

FIG. 12 shows in Panel A), an instrument 1200 tracked by computer vision in a method of the present disclosure; in Panel B), a variable diameter circle overlay placed on instrument tip in a method of the present disclosure; and in Panel C), a virtual ruler overlaid along instrument body of a method of the present disclosure.

FIG. 13 shows steps of a method of the present disclosure wherein different types of on-screen information can be displayed to the user to enable quick and accurate digital measurements or targeting. Virtual rulers are overlayed along the probe body and probe hook to enable quick measurements in Panels A and B. The user is able to rotate the virtual ruler about specific points to make measurements in directions not aligned with the probe body or probe hook in Panels C and D.

FIG. 14 shows steps of a method of the present disclosure wherein the relative orientation (a) between a probe body and a camera plane may be displayed on-screen to help the user adjust probe orientation

FIG. 15 shows steps of a method of the present disclosure wherein a probe tip point (point 1, FIG. 7) may be tracked across video frames to enable point-point on-screen measurements.

FIG. 16 shows steps of a method of the present disclosure wherein the tip of a micro-fracture awl is tracked using the image processing steps.

FIG. 17 shows steps of a method of the present disclosure wherein areas of interest are traced in the manner of FIG. 16 to include, for example, the anterior cruciate ligament (ACL) footprint during surgery.

FIG. 18 shows in Panels A, B, C, and D, embodiments of instruments according to the present disclosure each having a sleeve to improve computer vision based detection and tracking of the instruments.

FIG. 19 is an example of hardware that can be used to implement a computer vision based detection and tracking of the instruments according to the present disclosure.

Like reference numerals will be used to refer to like parts from Figure to Figure in the following detailed description. Any drawings herein are not shown to scale. Where dimensions are given in the text or figures, these dimensions are merely example values which could be used with one or more example implementations and do not limit the scope of the disclosed invention. Some embodiments of the disclosure are described herein with reference to the accompanying figures. The description, together with the figure, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure.

### DETAILED DESCRIPTION OF THE INVENTION

The present disclosure provides systems and methods for enabling real-time, digital on screen measurements and targeting during a medical procedure such as arthroscopic surgery.

Before the present invention is described in further detail, it is to be understood that the invention is not limited to the particular embodiments described. It is also to be understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

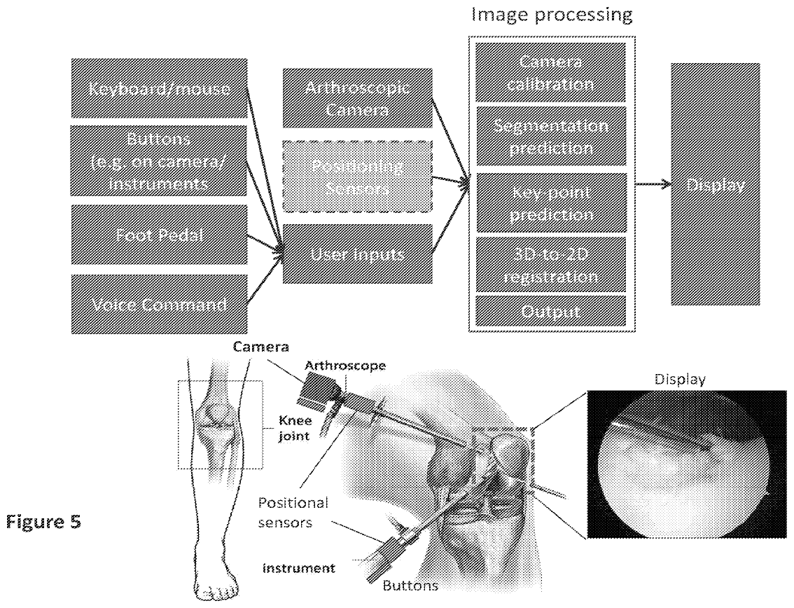

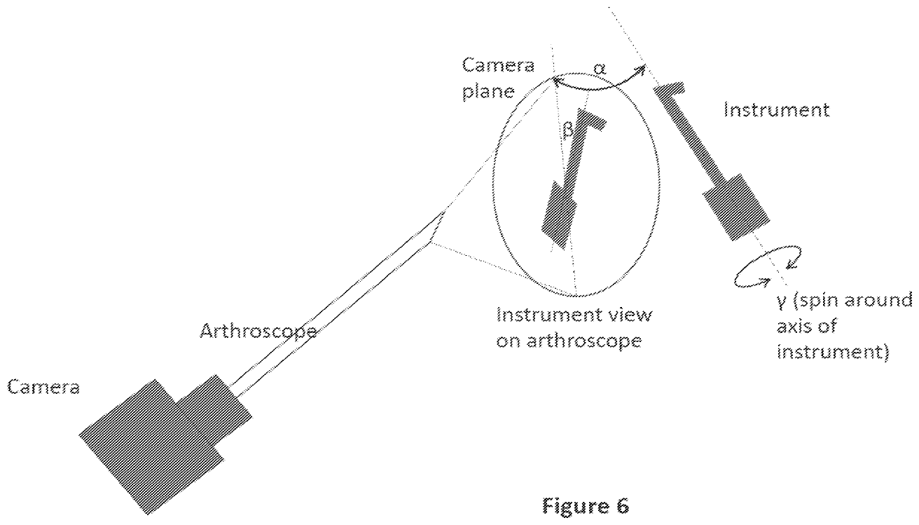

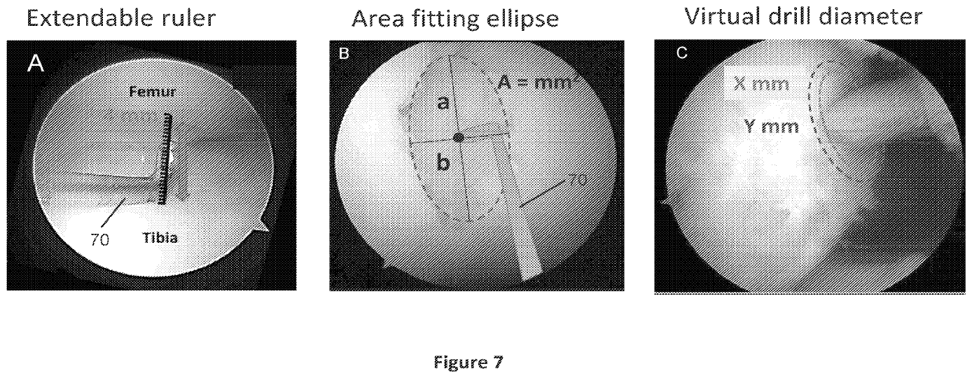

In one embodiment, the system includes a camera and light source attached to an arthroscope to capture the view inside the joint, an instrument (such as a hook probe, micro-fracture awl, drill guide etc.), a module to capture user inputs (e.g. via keyboard/mouse, physical buttons on camera/instrument, foot pedal, voice command etc.), optional positioning sensors placed on the arthroscope and/or the instrument, an image processing module, and a display to present information to the user (see FIG. 5). The positional sensors (if present) provide real-time data about relative angular orientation of the instrument with respect to the camera/camera plane in three-dimensional (3D) space (e.g., angles α, β, γ, see FIG. 6). These sensors can include any of a variety of sensors, including inertial measurement units which offer a simple and cost-effective means for measuring relative orientation of objects in 3D space. The image processing module, analyzes the video data coming from the arthroscopic camera and combines it with the user inputs, and orientation information from the positional sensors (if present) to track the six degree-of-freedom (6DOF) pose (location/orientation) of the instrument 70, and overlays appropriate visual information on the video data (see FIG. 7). The processed video data is then presented to the user via the display module (a computer screen, tablet, augmented reality glasses etc.). In embodiments of the invention where the positional sensors are not utilized, the requisite analysis/calculations are performed via image analysis methods alone.

The image processing module performs one or more of the following steps:
1. A calibration step to calculate camera matrix and account for distortion present in the arthroscopic images (e.g., radial fish-eye distortion).
2. Image segmentation using a combination of deep-learning and/or classical computer vision methods to detect the shape/contour of the instrument. This can be accomplished with specific segmentation models/algorithms trained to detect shape/contours of specific instruments. At the time of use, the specific models can be automatically selected based on user input about the instrument being used.
3. A key-point prediction step where 2D projected locations of pre-defined 3D key-points on the instrument are predicted on the arthroscopic image.
4. A 3D-to-2D registration process to predict the 6DOF pose of the instrument relative to the camera.
5. Data-output step that performs calculations and displays appropriate visual information on the processed video presented to the user.

### Step 1—Camera Calibration:

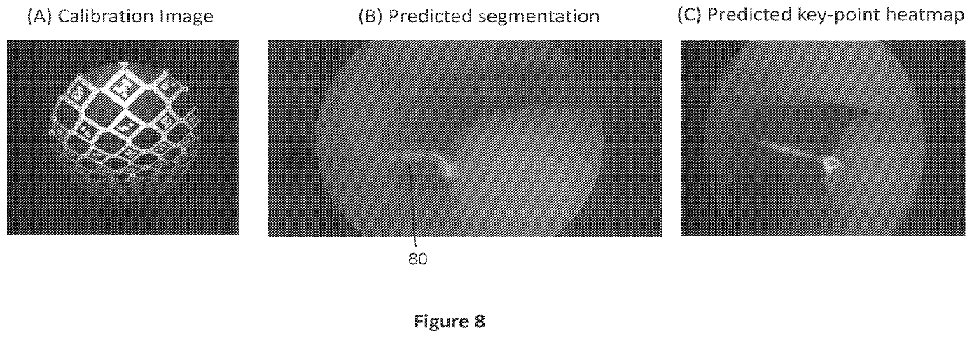

This step is performed to calculate camera intrinsic/extrinsic matrix, and perform distortion correction. Any of the known approaches to distortion correction, such as that described in [Ref. 12,13], can be used. A pre-saved file specific to the camera/lens system can be used for this calibration step. Alternatively, an intraoperative calibration step can be performed by imaging a calibration image such as a grid pattern (FIG. 8 Panel A).

### Step 2—Segmentation of Instrument:

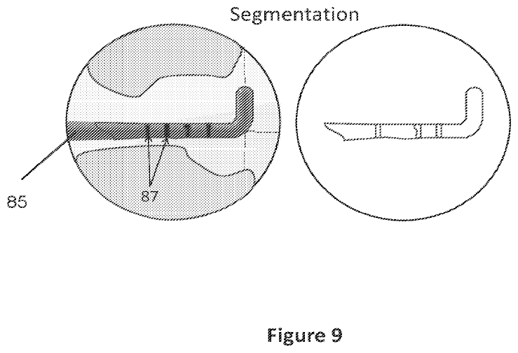

In the second step, computer vision models are trained to detect and predict the contour of the instrument 80 on the video frames. Examples of models that can be used include deep learning based semantic and instance segmentation architectures such as Fully Convolutional Network (FCN), U-Net, DeepLab v3, Mask R-CNN, SegFormer, etc. [Ref. 9,10]. The output of such models is a prediction of image pixels that correspond to the class of the interest (in this case the instrument). This model prediction is then used to create a predicted segmentation mask (FIG. 8 Panel B). The models can be trained to segment either just the outer profile of the instrument 80 or both the outer profile of the instrument 85 and internal features such as the edges of bands 87 (see FIG. 9). In some embodiments of the invention, deep learning-based methods may be used to predict segmentation mask for the outer profile of the instrument, and then used to crop out the instrument from the image. Other computer vision methods, such as canny edge detection, Sobel filter, etc. can then be applied to the cropped instrument image to detect the edges of internal features such as bands.

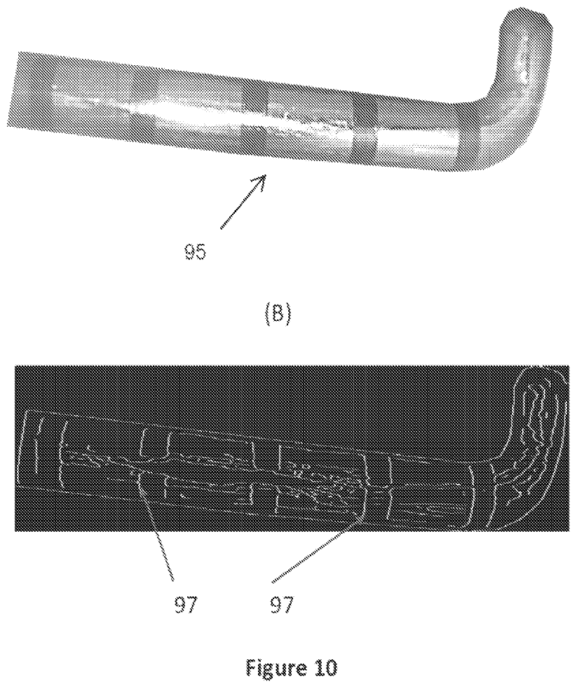

In one exemplary embodiment of the invention, a Mask R-CNN instance segmentation model, was trained via the opensource Detectron2 framework [Ref. 11], by providing several manually labelled images. Two class labels were included, (1) hook probe, and (2) tissue. Although the primary class of interest was the hook probe, including the background “tissue” class (which surrounds the hook probe) improved detection of the primary class. Images were grouped into a training/validation set in 80:20 ratio (separate image sets used for testing), and trained for 25 epochs with default augmentation options in Detectron2 (random brightness and random crop). The trained model outputs predicted segmentation masks of the probe hook 95 on the video frames. The predicted segmentation masks were then used to crop the image, and Sobel filter applied to detect the probe band edges 97 (see FIG. 10), using classical computer vision to detect probe band edges [e.g., canny edge detection http://bigwww.epfl.ch/demo/ip/demos/edgeDetector/].

The segmentation mask can be used to improve the key-point prediction in Step 3. This can be done by either by providing the mask as another input channel along with the original image, or by using the image cropped with the segmentation mask an input for the key-point prediction.

### Step 3—Key-Points Prediction:

In this step, a model predicts the projected locations of known 3D key-points of the instrument on the 2D arthroscopic image. Key-points are generally 3D points distributed on the instrument surface in a region likely to be in the view of the camera, and adequately spread-out to maximize pose estimation accuracy in the step 4. In the exemplary embodiment of the invention, the original image cropped with segmentation mask from step 2 is presented as an input to an Hourglass network based deep CNN, which outputs a heatmap representing the prediction confidence for the key-point locations (FIG. 8 Panel C). The centroid of the heatmap is used as the predicted 2D projection of the known 3D key-point on the image.

### Step 4—Prediction of Instrument 6DOF Pose:

The 6DOF pose of the instrument relative to the camera is predicted via a 3D-to-2D registration process. First Perspective-n-Point (PnP) with RANSAC algorithm is applied to the 3D key-points and their 2D predictions obtained from step 3, to obtain an initial guess for the 6DOF pose of the instrument relative to the camera. Next, an edge-based heuristic procedure is used to minimize differences between the segmented mask of step 3, and 2D projection of the instrument placed at the initial pose. The initial pose of the instrument is perturbed until a pose that minimizes differences (L1 or L2 norm) between the segmented mask and 2D projection of the instrument is identified. This final pose is used as the predicted 6DOF pose for the instrument.

### Step 5—Data-Output:

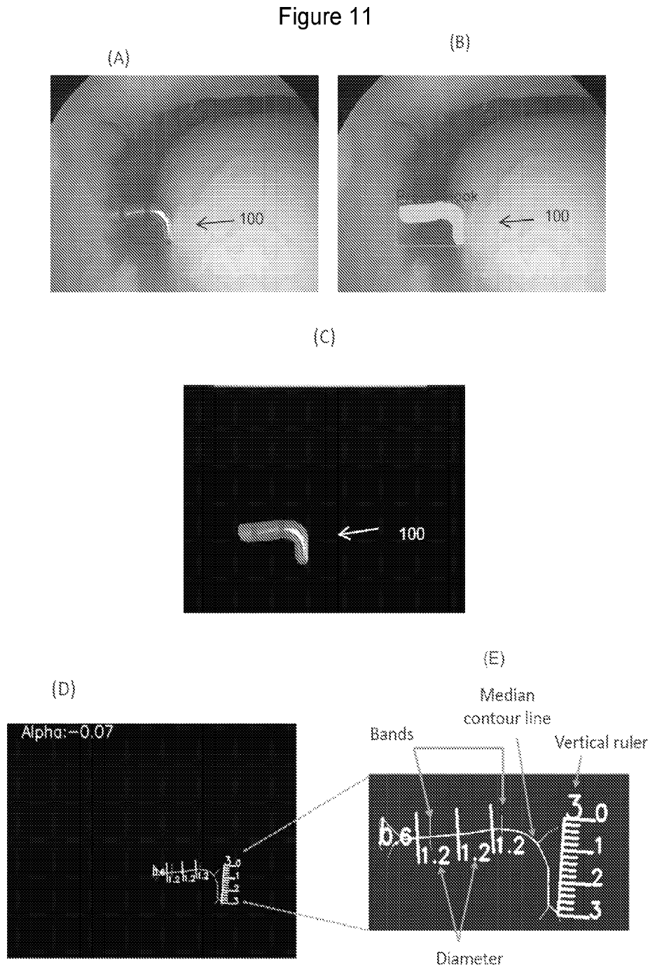

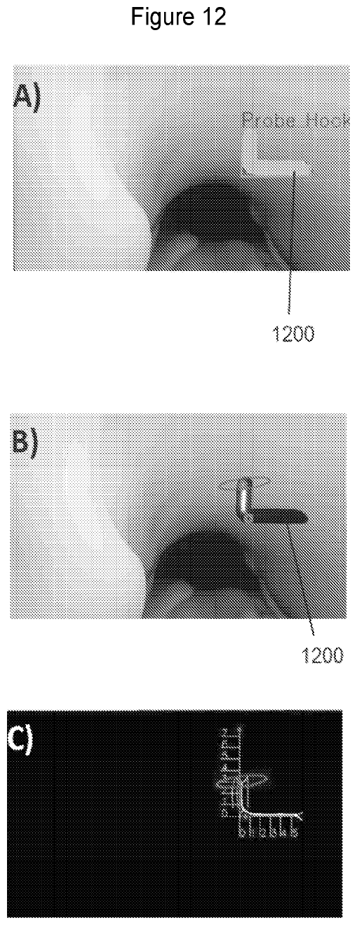

Different types of on-screen information, such as virtual rulers, area fitting circle/ellipses, drill diameters etc. can be displayed to the user to enable quick and accurate digital measurements or targeting (see FIGS. 11 and 12). The location and appropriate scaling of these overlays is enabled by coordinate transformation based on 6DOF position of the instrument relative to the camera predicted in step 4, and knowledge of 3D geometry of the instrument. In FIG. 11, Panel A shows the original video frame with a probe 100; Panel B shows the detection of the probe hook, and predicted segmentation mask; Panel C shows the image cropped using predicted segmentation; and Panels D and E show the detections/calculations. FIG. 12 shows in Panel A), an instrument 1200 tracked by computer vision; in Panel B), a variable diameter circle overlay placed on the instrument tip; and in Panel C), a virtual ruler overlaid along instrument body.

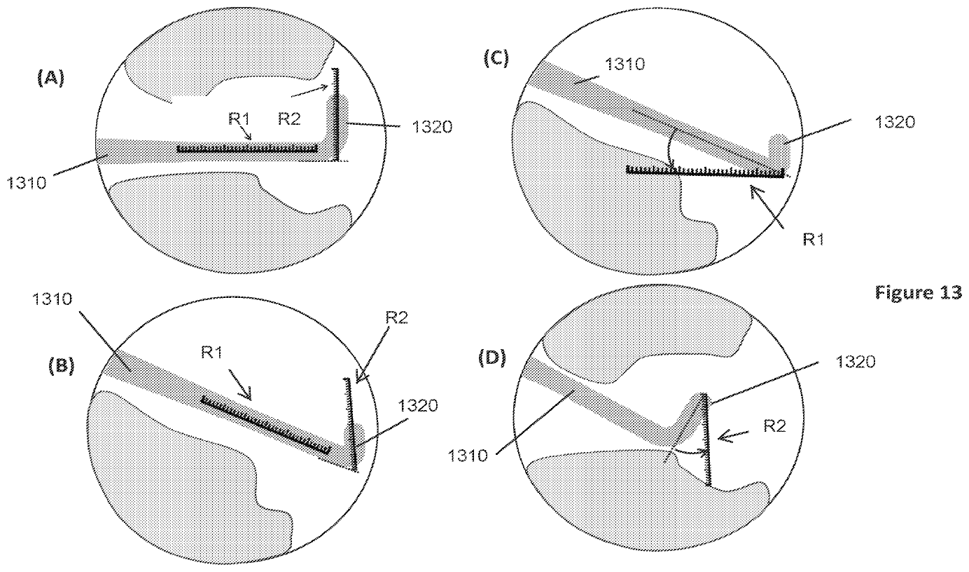

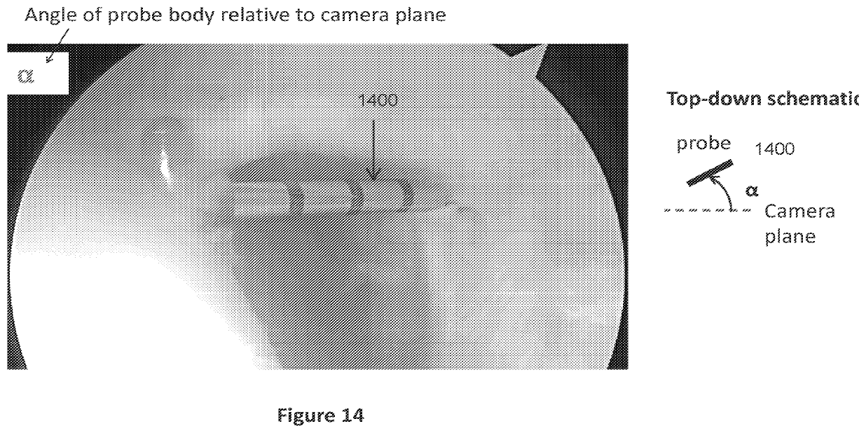

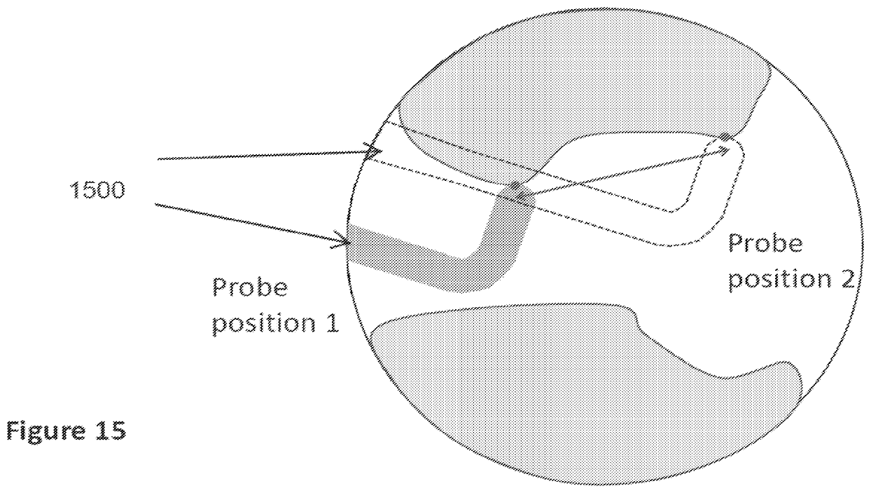

In one embodiment of the invention, virtual rulers R1, R2 are overlayed along the probe body 1310 and probe hook 1320 to enable quick measurements (see FIG. 13 Panels A, B). In yet other embodiments of the invention, the user is able to rotate the virtual ruler about specific points to make measurements in directions not aligned with the probe body 1310 or probe hook 1320 (see FIG. 13 Panels C, D). In other embodiments of the invention, the relative orientation (a) between probe body 1400 and camera plane may be displayed on-screen to help the user adjust probe orientation if they so desire (see FIG. 14). In yet another embodiment of the invention, the probe tip of the probe 1500 may be tracked across video frames to enable point-point on-screen measurements (see FIG. 15). In this embodiment, computer vision methods can be used to compensate for minor camera movements while probe is moved by user from one location to the next within the camera field of view.

It is also important to provide users the ability to control the display of the visual information, such as the ability to turn on/off the display, rotate or extend the virtual ruler, change the size of virtual circles/ellipses start or stop probe tip tracking for point-point measurements etc. During arthroscopy, the user (surgeon) typically holding an instrument in one hand and the arthroscope in the other hand. Therefore, display control should ideally be provided via a hands-free approach (e.g., through voice control, foot pedal) or by placement of buttons/joystick on the instrument handle or the arthroscope.

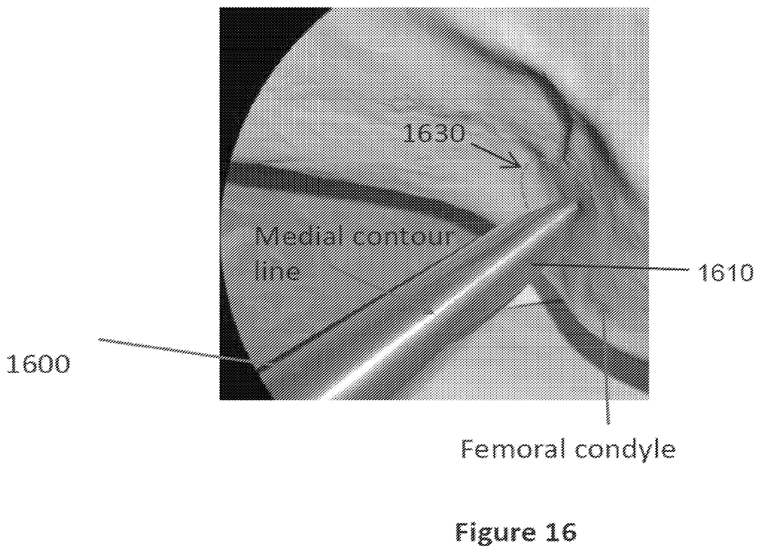

While the exemplary embodiment of the invention shown in FIGS. 11 and 12 pertains to detection/tracking of a hook probe, in further embodiments of the invention, instruments used for marking and/or drilling insertion sites for placement of fixation devices or reconstructive grafts can be tracked using computer vision. This is further illustrated with the example of an anterior cruciate ligament (ACL) reconstruction surgery. During ACL reconstruction, surgeons need to select a graft of certain diameter (e.g., 8-12 mm), and mark or otherwise plan the appropriate location for a drill hole on the femur and tibia where anchors for the ACL graft are to be placed. The drill hole should be in an appropriate anatomical location within the native footprint, and sufficiently distant from edges of the bone, especially on the posterior femoral condyle, to avoid blowout failure of the bone. To aid in this targeting decision, in one embodiment of the invention, computer vision methods are used to detect and track instruments used to mark the drill hole location (such as a microfracture awl, electrocautery wand, guide wire etc.). For example, in the embodiment of FIG. 16, the tip 1610 of a micro-fracture awl 1600 is tracked using the image processing steps 14 of the invention. A virtual circle 1630 corresponding to the drill diameter (or graft size) is then shown near the tip of the instrument in the arthroscopy video. Coordinate transformations based on 6DOF instrument pose prediction is used to appropriately display the size and shape of the virtual circle. The virtual circle 1630 helps the surgeon visualize where the edges of planned drill hole would be relative to the bony anatomy, prior to the start of the drilling step.

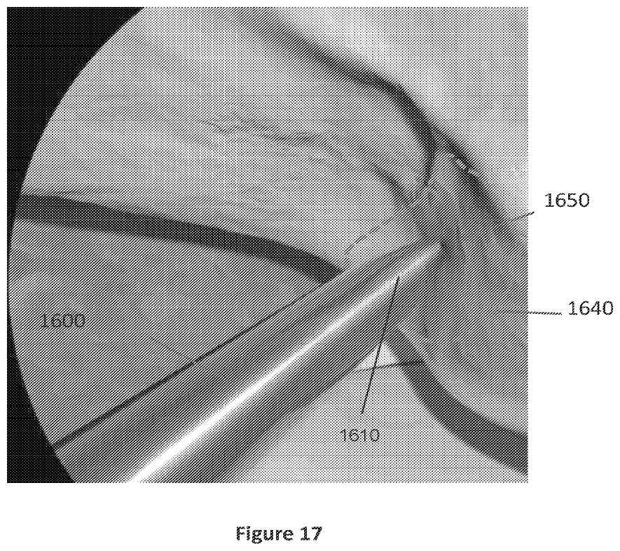

In further embodiments of the invention, once the desired arthroscopic view is achieved, the instrument tip can be tracked and used as a virtual pen/brush by the surgeon to trace out areas of interest, such as a virtual “ACL footprint” 1640 traced by using awl as a pen/pointer. During this process, the arthroscope should not be moved excessively to maintain virtual markings at correct location relative to the underlying anatomy. Areas of interest that could be traced in this manner, include for example the ACL footprint 1640. Centroids of footprint 1650 (or different quadrants) can then be automatically displayed to serve as potential targets for the drilling location (see FIG. 17).

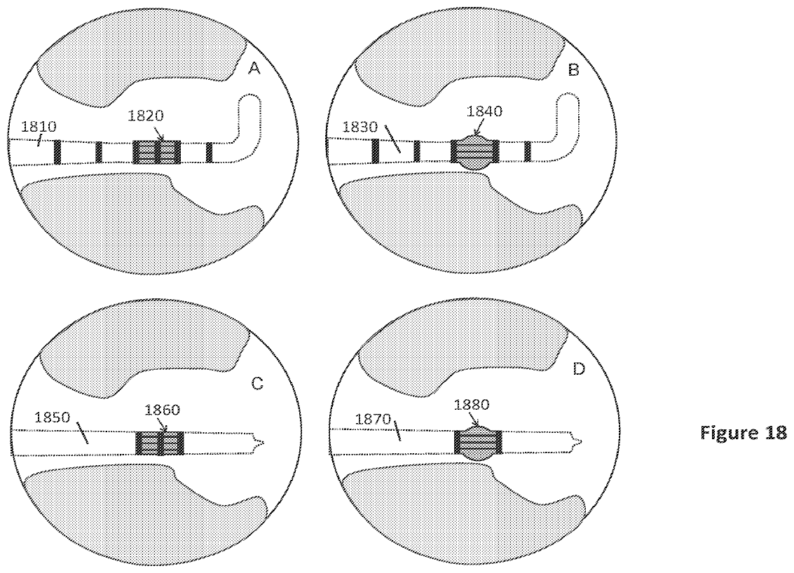

In yet further embodiments of the invention, the generalizability of computer vision-based detection and tracking of instruments may be improved by adding specific features or making modifications to instruments such that: a) they are easier to detect, and b) making these features common across instruments so that the same computer vision method/s can be used to detect/track various instruments without explicitly retraining the computer vision model for each instrument. In one embodiment, this is achieved by adding a sleeve 1820 to a hook probe 1810 (see FIG. 18, Panel A). In another embodiment, this is achieved by adding a sleeve 1840 to a hook probe 1830 (see FIG. 18, Panel B). In another embodiment, this is achieved by adding a sleeve 1860 to an awl 1850 (see FIG. 18, Panel C). In another embodiment, this is achieved by adding a sleeve 1880 to an awl 1870 (see FIG. 18, Panel D). The sleeve can be a separate component, or made integral part of the instrument. The sleeve in turn contains features/and or textures such as parallel lines, vertical bands, a central spherical shape etc., to aid in computer vision-based detection of the sleeve (and by extension the instrument). A computer vision model is then trained to detect/track the sleeve. Thereafter, the sleeve can be added to any instrument and used in concert with the pre-trained computer vision model for instrument detection and tracking, without retraining the computer vision model for the new instrument. By adding the sleeve at a known location on the instrument and utilizing coordinate transformation methods, the relationship between the sleeve location (such as sleeve center) as detected by computer vision, and another point on the instrument (such as instrument tip) can be calculated. Here again, the use of orientation sensors can aid in more accurate coordinate transformation from sleeve to another location on the instrument.

In some alternative embodiments of the invention, smoothening/curve fitting operations may be used to refine the predicted segmentation.

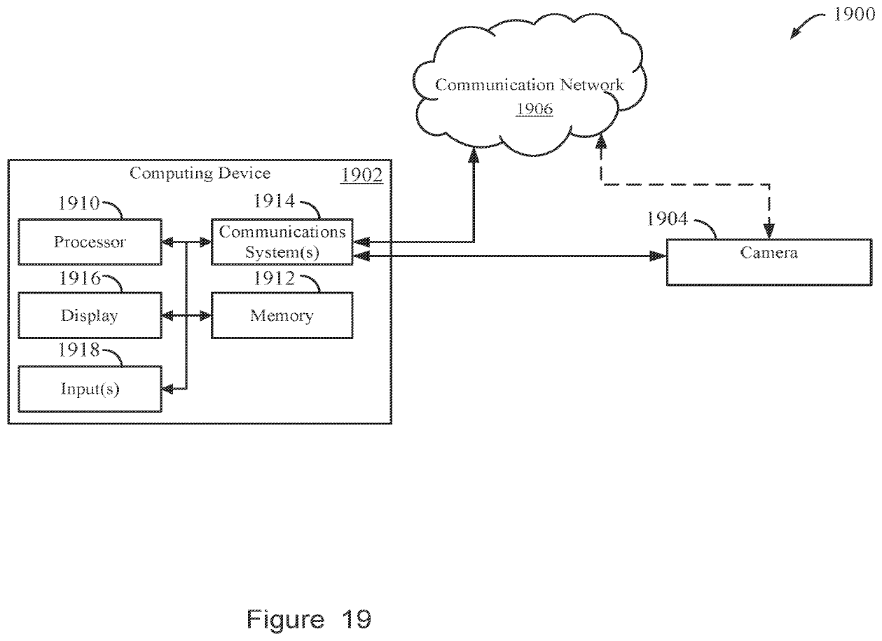

FIG. 19 is a schematic diagram illustrating an example computer system 1900, which can be used to implement the processes and systems described above. As shown, the computer system 1900 can include a computing device 1902, a camera 1904, and a communication network 1906.

In some cases, the camera 1904 can be a camera sized and configured to capture an image of an internal area of a subject. A “subject” is a mammal, preferably a human. For example, the camera 1904 can be one of an arthroscope, an endoscope, a laparoscope, a cardioscope, or any other known camera for capturing internal images of a subject. In some cases, the camera 1904 is directly connected to the computing device 1902 (e.g., via a wired connection). In some cases, the camera can communicate images to the computing device 1902 through the communications network 1906.

As further shown in FIG. 19, the computing device 1902 can include a processor 1910, memory 1912, communications systems 1914, a display 1916, and input(s) 1918. In some embodiments, processor 1910 can be any suitable hardware processor or combination of processors, such as a central processing unit (CPU), a graphics processing unit (GPU), an accelerated processing unit (APU), an application specific integrated circuit (ASIC), a field-programmable gate array (FPGA), a combination thereof, etc.

In some embodiments, memory 1912 can include any suitable storage device or devices that can be used to store instructions, values, configurations, etc., that can be used, for example, by processor 1910 to process image data received from the camera 1904. For example, memory 1912 can include software which can be used by operators to implement processes for providing visualization for real-time image data received from camera 1904, machine learning models for processing images obtained by camera 1904 and calculating parameters therefrom, 2D projections of medical instruments (e.g., an awl, or a hooked probe, as described above) to be detected in the images from the camera 1904, 3D models of the medical instruments to be detected in images received from the camera 1904, pre-saved calibration file specific to the camera for use in distortion correction, known dimensions of the medical instrument, etc. Memory 1912 can include any suitable volatile memory, non-volatile memory, storage, or any suitable combination thereof. The memory 1912 can comprise a non-transitory computer readable medium including instructions for implementing the systems and processes described herein. For example, memory 1912 can include random access memory (RAM), read-only memory (ROM), electronically-erasable programmable read-only memory (EEPROM), one or more flash drives, one or more hard disks, one or more solid state drives, one or more optical drives, etc. In some embodiments, memory 1912 can have encoded thereon a computer program for implementing the processes described herein.

In some embodiments, communications systems 1914 can include any suitable hardware, firmware, and/or software for performing wired or wireless communications (e.g., with a user device, with camera 1904, etc.) over communication network 1906 and/or any other suitable communication networks. For example, communications systems 1914 can include one or more transceivers, one or more communication chips and/or chip sets, etc. In a more particular example, communications systems 1914 can include hardware, firmware and/or software that can be used to establish a Wi-Fi connection, a Bluetooth connection, a cellular connection, an Ethernet connection, etc. In some examples, image data can be streamed to the computing device 1902 from the camera 1904 through communications system 1914 (e.g., via the communication network 1906 or through a direct wired connection between the camera 1904 and the computing device 1902. In some examples, communications system 1914 can be in communication with the communications network 1906 and can communicate information thereto, receive instructions or configurations, receive firmware or software updates, etc.

In some embodiments, the display 1916 can include any suitable display device, such as a computer monitor, a touchscreen, a television, a projector, a smart phone, a virtual reality headset, augmented reality goggles, etc. In some cases, a display can be remote relative to the computing device. For example, a processor and memory of a computing device can be hosted remotely (e.g., on a cloud server) and a display can receive visualizations and images from the computing device over a network connection. In some embodiments, inputs 1918 can include any suitable input devices and/or sensors that can be used to receive user input, such as a keyboard, a mouse, a touchscreen, a microphone, a camera, a foot pedal, etc.

Thus, the present invention provides systems and methods for enabling real-time, digital on screen measurements and targeting during a medical procedure such as arthroscopic surgery.

### References

1. Shah, N. V., et al., “Demographics and rates of surgical arthroscopy and postoperative rehabilitative preferences of arthroscopists from the Arthroscopy Association of North America (AANA)”, J Orthop, 2018. 15(2): p. 591-595.
2. Parikh, S. N., et al., “Complications of medial patellofemoral ligament reconstruction in young patients”, Am J Sports Med, 2013. 41(5): p. 1030-8.
3. Elias, J. J. and A. J. Cosgarea, “Technical errors during medial patellofemoral ligament reconstruction could overload medial patellofemoral cartilage: a computational analysis”, Am J Sports Med, 2006. 34(9): p. 1478-85.
4. Samitier, G., et al., “Failure of Anterior Cruciate Ligament Reconstruction,” Arch Bone Jt Surg, 2015. 3(4): p. 220-40.
5. Wright, R. W., et al., “Descriptive epidemiology of the Multicenter ACL Revision Study (MARS) cohort”, Am J Sports Med, 2010. 38(10): p. 1979-86.
6. Mather, R. C., 3rd, et al., “Societal and economic impact of anterior cruciate ligament tears”, J Bone Joint Surg Am, 2013. 95(19): p. 1751-9.
7. Tanaka, M. J., et al., “Arthroscopic Patellofemoral Measurements Can Reliably Assess Patellar Instability”, Arthroscopy, 2021.
8. Xu Han, Peizhao Wang, Jinyang Yu, Xiao Wang, and Honglue Tan, “Arthroscopic piecrusting release of the posteromedial complex of the knee for surgical treatment of medial meniscus injury”, BMC Musculoskeletal Disordersb(2020) 21:301.
9. https://paperswithcode.com/task/semantic-segmentation
10. Rabi Sharma, Muhammad Saqib, C. T. Lin, Michael Blumenstein, “A Survey on Object Instance Segmentation”, SN Computer Science volume 3, Article number: 499(2022).
11. https://detectron2.readthedocs.io/en/latest/tutorials/getting_started.html
12. Nicole A. Kallemeyn, Nicole M. Grosland, Vincent A. Magnotta, James A. Martin, Douglas R. Pedersen, “Arthroscopic lens distortion correction applied to dynamic cartilage loading”, The lowa Orthopaedic Journal, 2007.
13. Aashay L. Kekatpure, Arnold Adikrishna, Ji-Ho Sun, Gyeong-Bo Sim, Jae-Myung Chun, In-Ho Jeon, “Comparative analysis of visual field and image distortion in 30° and 70° arthroscopes”, Knee Surg Sports Traumatol Arthrosc (2016) 24:2359.2364.
14. Barbara Postolka, Renate Lista, Benedikt Thelen, Pascal Schutz, William R. Taylor, Guoyan Zheng, “Evaluation of an intensity-based algorithm for 2D/3D registration of natural knee videofluoroscopy data”, Medical Engineering & Physics, Volume 77, March 2020, Pages 107-113.
15. Daniel Toth, Shun Miao, Tanja Kurzendorfer, Christopher A. Rinaldi, Rui Liao, Tommaso Mansi, Kawal Rhode, Peter Mountney, “3D/2D model-to-image registration by imitation learning for cardiac procedures”, International Journal of Computer Assisted Radiology and Surgery volume 13, pages 1141-1149 (2018).
16. https://docs.opencv.org/3.4/d3/dc0/group__imgproc__ shape.html#ga17ed9f5d79ae97bd4c7cf18403e1689a

The citation of any document or reference is not to be construed as an admission that it is prior art with respect to the present invention.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment”, “in other embodiments”, “in some embodiments”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

What is claimed is:

1. A system for performing digital measurements during a medical procedure in a subject, the system comprising:
a camera positionable to capture image data corresponding to a treatment site in a subject;
a display;
an instrument dimensioned to be placed at the treatment site, the instrument including at least a first section having a first dimension between a first boundary and a second boundary;
at least one memory, the at least one memory having stored thereon a configuration corresponding to the instrument, the configuration including the first dimension; and
at least one processor in electrical communication with the camera and the at least one memory, the at least one processor configured to execute instructions stored in the at least one memory to:
(i) receive real-time image data from the camera, the real-time image data including at least a portion of the instrument,
(ii) detect, in the real-time image data, a profile of the instrument,
(iii) identify, on the instrument, the first boundary and the second boundary,
(iv) determine a real-time measurement between a first set of points of the real-time image data, the real-time measurement being determined based at least in part on the first dimension,
(v) generate a visualization based on the real-time measurement, and
(vi) display, at the display, the visualization and the real-time image data.

2. The system of claim 1, wherein the first dimension is a diameter of the instrument.

3. The system of claim 1, wherein the first boundary and the second boundary are visually-identifiable markings along the instrument.

4. The system of claim 3, wherein the instrument includes a removable sleeve, wherein the first boundary and the second boundary are defined on the removable sleeve.

5. The system of claim 1, wherein the visualization includes a first digital ruler, wherein the first digital ruler is overlaid onto the real-time image data at the display.

6. The system of claim 1, wherein the instrument comprises an awl.

7. The system of claim 1, wherein the at least one processor is further configured to execute instructions stored in the at least one memory to identify, in the real-time image data, a distal tip of the instrument.

8. The system of claim 7, wherein a placement of the visualization on the display is based at least in part on a position of the distal tip.

9. The system of claim 8, wherein the real-time measurement is a measurement of a radius extending from the distal tip.

10. The system of claim 7, wherein the first set of points comprises a first point corresponding to a position of the distal tip at a first time, and a second point corresponding to a position of the distal tip at a second time, wherein the first measurement is a distance between the first point and the second point.

11. The system of claim 1, wherein the instrument includes a first linear portion and a second linear portion, the second linear portion being substantially perpendicular to the first linear portion, the second portion defining a second dimension between a first end and a second end, wherein the configuration includes the second dimension, and wherein the at least one processor is further configured to execute instructions stored in the at least one memory to determine a second real-time measurement between a second first set of points of the real-time image data, the second real-time measurement being determined based at least in part on the second dimension.

12. The system of claim 1, wherein the at least one processor is further configured to execute instructions stored in the at least one memory to receive an instrument input, and, based on the instrument input, select, from a plurality of instrument configurations stored in the at least one memory, the configuration.

13. The system of claim 1, wherein the medical procedure is an arthroscopic knee procedure.

14. The system of claim 1, wherein the instrument includes a first three-dimensional key point, wherein determining the real-time measurement includes predicting a location of the first three-dimensional key point on the real-time image.

15. The system of claim 1, wherein determining the real-time measurement includes minimizing a difference between the profile of the instrument and a two-dimensional projection of a three-dimensional model of the instrument, and, based on the two-dimensional projection, determining a pose of the instrument relative to the camera, wherein the real-time measurement is based on the pose.

16. The system of claim 15, wherein the pose of the instrument is a 6 degree of freedom pose.

17. A method for performing digital measurements during a medical procedure in a subject, the method comprising:
receiving a first image, the first image including a visual representation of a first instrument and a tissue of a subject;
detecting, within the first image, a profile of the first instrument, including an outer edge of the first instrument;
comparing the profile of the first instrument to a two-dimensional projection of a three-dimensional model of the first instrument to determine a pose of the instrument relative to the camera;
identifying a first key feature of the instrument;
determining, based on the first key feature and the pose, a first dimension of the first image;
generating an overlay including a first visualization based at least in part on the first dimension; and
outputting, at a display, the overlay and the first image.

18. The method of claim 17, wherein detecting a profile of the first image includes inputting the first image into a trained machine learning model, the trained machine learning model being trained on a first plurality of images, each of the first plurality of images including a first label identifying an instrument within the image, and a second label identifying a tissue within the image.

19. The method of claim 17, wherein the first image is received in real-time from an arthroscope.

20. The method of claim 17, further comprising providing, prior to receiving the first image, a plurality of two-dimensional projections of the instrument, and selecting a first two-dimensional projection of the plurality of two-dimensional projections based on a similarity between the profile of the first instrument and the first two-dimensional projection, wherein performing the smoothing operation includes altering the profile to increase a similarity between the profile and the first two-dimensional projection.

21. The method of claim 17, wherein the first key point is a first band positioned along the instrument, wherein the first dimension is a known distance between the first band and a second band positioned along the first instrument.

22. The method of claim 17, wherein the first visualization is a digital ruler, wherein a scale of the digital ruler is determined at least in part based on the first dimension.

23. The method of claim 17, further comprising:
receiving a second image, the second image including a visual representation of the first instrument and a tissue of the subject;
identifying, in the second image, the first key point;
determining, based at least partially on the first dimension, a measurement between a position of the first key point in the first image, and the first key point in the second image; and
outputting, at the display, the measurement.

24. The method of claim 17, wherein the medical procedure is an arthroscopic knee procedure.

25. The method of claim 17, wherein the tissue is tissue of a joint.

26. The method of claim 25, wherein the joint is a knee.

27. A computer system comprising instructions stored on a non-transitory computer readable medium to cause at least one processor on a computer to:
receive a first image, the first image including a visual representation of a first instrument and a tissue of a subject;
detect, within the first image, a profile of the first instrument, including an outer edge of the first instrument;
determine a predicted pose of the first instrument relative to the camera based on the profile;
select a first two-dimensional projection of a three-dimensional model of the instrument based on the predicted pose;
determine an actual pose of the instrument based at least in part on a comparison between the profile and the first two-dimensional projection;
determine, based on the actual pose, a first dimension of the first image;
generate an overlay including a first visualization based at least in part on the first dimension; and
output, at a display, the overlay and the first visualization.

28. The computer system of claim 27, wherein the tissue is tissue of a joint.

29. The computer system of claim 28, wherein the joint is a knee.

30. The computer system of claim 27, wherein the instructions stored on the non-transitory computer readable medium further cause at least one processor on a computer to:
select, based on the comparison between the profile and the first two-dimensional projection, a second two-dimensional projection,
minimizing a difference between the profile and the second two-dimensional projection,
wherein the actual pose corresponds to a model pose of the second two-dimensional projection.

31. A method for performing digital measurements during a medical procedure in a subject, the method comprising:
receiving a first image, the first image including a visual representation of a first instrument and a tissue of a subject;
detecting, within the first image, a profile of the first instrument, including an outer edge of the first instrument;
performing a smoothening operation on at least a first portion of the outer edge, wherein the smoothening operation at least partially alters a profile of the first instrument;
identifying a first key feature of the instrument;
determining, based on the first key feature, a first dimension of the first image;
generating an overlay including a first visualization based at least in part on the first dimension; and
outputting, at a display, the overlay and the first image.

32. The method of claim 31, wherein detecting a profile of the first image includes inputting the first image into a trained machine learning model, the trained machine learning model being trained on a first plurality of images, each of the first plurality of images including a first label identifying an instrument within the image, and a second label identifying a tissue within the image.

33. The method of claim 31, wherein the first image is received in real-time from an arthroscope.

34. The method of claim 31, further comprising providing, prior to receiving the first image, a plurality of two-dimensional projections of the instrument, and selecting a first two-dimensional projection of the plurality of two-dimensional projections based on a similarity between the profile of the first instrument and the first two-dimensional projection, wherein performing the smoothing operation includes altering the profile to increase a similarity between the profile and the first two-dimensional projection.

35. The method of claim 31, wherein the first key point is a first band positioned along the instrument, wherein the first dimension is a known distance between the first band and a second band positioned along the first instrument.

36. The method of claim 31, wherein the first visualization is a digital ruler, wherein a scale of the digital ruler is determined at least in part based on the first dimension.

37. The method of claim 31, further comprising:
receiving a second image, the second image including a visual representation of the first instrument and a tissue of the subject;
identifying, in the second image, the first key point;
determining, based at least partially on the first dimension, a measurement between a position of the first key point in the first image, and the first key point in the second image; and
outputting, at the display, the measurement.

38. The method of claim 31, wherein the medical procedure is an arthroscopic knee procedure.

39. The method of claim 31, wherein the tissue is tissue of a joint.

40. The method of claim 39, wherein the joint is a knee.

41. A computer system comprising instructions stored on a non-transitory computer readable medium to cause at least one processor on a computer to:
receive a first image, the first image including a visual representation of a first instrument and a tissue of a subject;
detect, within the first image, a profile of the first instrument, including an outer edge of the first instrument;
perform a smoothening operation on at least a first portion of the outer edge, wherein the smoothening operation at least partially alters a profile of the first instrument;
identify a first key feature of the instrument;
determine, based on the first key feature, a first dimension of the first image;
generate an overlay including a first visualization based at least in part on the first dimension; and
output, at a display, the overlay and the first visualization.

42. The system of claim 41, wherein the tissue is tissue of a joint.

43. The system of claim 42, wherein the joint is a knee.
